(19) United States
(12) Patent Application Publication
(10) Pub. No.: US 2023/0390325 A1
(43) Pub. Date: Dec. 7, 2023

(54) Oral and Nasal Wash

(71) Applicant: Florence Pun, San Francisco, CA (US)

(72) Inventor: Florence Pun, San Francisco, CA (US)

(21) Appl. No.: 18/111,464

(22) Filed: Feb. 17, 2023

Related U.S. Application Data
(60) Provisional application No. 63/348,241, filed on Jun. 2, 2022.

(51) Int. Cl. A61K 31/785; A61K 9/08; A61K 9/00

(52) U.S. Cl. CPC A61K 31/785; A61K 9/08; A61K 9/0043

(57) ABSTRACT

Oral and nasal wash containing poly-gamma-glutamic acid (γ-PGA), a natural and nontoxic biopolymer that kills bacteria, is a safe and effective approach to killing bacteria in wounds and oral/nasal cavities. Administering aqueous formulations of γ-PGA can prevent infections in the nasopharyngeal cavity and in the oral cavity. The present invention is particularly suited for treating patients with medical conditions such as cleft lip and palate who are susceptible to bacterial infections and diseases.

### REFERENCE TO RELATED APPLICATION

This application claims priority under 35 U.S.C. § 119(e) to co-pending application No. 63/348,241 filed Jun. 2, 2022, which is incorporated herein by reference.

### FIELD OF INVENTION

The present invention relates to a natural non-toxic antibacterial oral and nasal rinse or wash which contains poly-gamma-glutamic acid (γ-PGA).

### BACKGROUND OF THE INVENTION

Saline mouth and nasal washes are typically used for daily cleansing and hydration of oral and nasal cavities and sinuses. They are also used after oral and nasal surgical procedures. However, these saline washes are unable to sufficiently kill bacteria in wounds and oral/nasal cavities.

Alcohol oral and nasal washes effectively kill bacteria but are not used after surgical procedures. Alcohols may irritate oral and nasal wounds and nasal tissues and are harmful when ingested. For example, chlorhexidine cannot only sting wounds after surgical oral procedures but is also known to stain teeth and dental mucosa, as well as alternate taste perception. Children are often prone to accidentally swallowing chemical mouthwashes. These products are often essential to kill bacteria that cannot be killed from other dental health routines, such as teeth brushing. Individuals who improperly brush their teeth, or young children who still need to learn to brush their teeth properly may rely on natural mouthwashes to kill additional bacteria. Additionally, nasal washes are often used to remove debris, pollen, and mucus that harbor many species of bacteria. Nasal washes are effective in removing bacteria to prevent allergies and viruses to prevent influenza and even coronaviruses. Alcohol saline nasal washes cannot be used as the products would damage the nasal mucosa and leave a painful, burning sensation in the nose. Presently, there is no ideal solution to effectively kill many forms of bacteria in patients without the incorporation of toxic chemicals and alcohol.

### SUMMARY OF THE INVENTION

The present invention is based in part on the recognition that an oral and nasal wash containing poly-gamma-glutamic acid (γ-PGA), a natural and nontoxic biopolymer that kills bacteria, can serve as a safe and effective approach to killing bacteria in wounds and oral/nasal cavities. In one aspect, the invention is directed to a method of preventing infections in the nasopharyngeal cavity (sinuses) that includes administering a pharmaceutically effective amount of γ-PGA. In another aspect, the invention is directed to a method of preventing infections in the oral cavity that includes administering a pharmaceutically effective amount of γ-PGA. The present invention is particularly suited for treating patients with medical conditions, such as cleft lip and palate, who are susceptible to bacterial infections and diseases.

### DESCRIPTION OF PREFERRED EMBODIMENTS

The oral/nasal wash of the present invention can be used to kill the hundreds of species of bacteria and biofilms that harbor in a person's oral and nasal cavities. γ-PGA is a naturally occurring biopolymer made up of repeating units of L-glutamic acid and/or D-glutamic acid. γ-PGA has five different conformations: α-helix, β-sheet, helix-to-random coil transition, random coil, and enveloped aggregate. A specific conformation forms when environmental conditions, including pH, ionic strength, and polymer concentration are altered. The compound is non-toxic (alcohol-free), biodegradable, and non-immunogenic and can act as a lubricant, and can be used in skin products as a hydrophilic humectant to hydrate and moisturize the skin. γ-PGA has also been used in the food and medical industries. In the food industry, γ-PGA has been used as a food supplement, osteoporosis-preventing agent, texture enhancer, cryoprotectant, and oil-reducing agent.

In medicine, γ-PGA has been used as metal chelators, drug carriers, and biological adhesives and has taken part in tissue engineering. Peptidoglycan-bound γ-PGA can prevent phage infections and antibodies from accessing bacteria. Staphylococcus epidermidis synthesizes γ-PGA to protect against antimicrobial peptides and prevent phagocytosis, ultimately preventing virulence. γ-PGA has also been released into the environment to isolate toxic metal ions, and protect against harmful conditions. Lastly, γ-PGA can improve biofilm formation and helps promote the absorption of essential nutrients from the environment. Studies have shown that solutions containing γ-PGA can kill bacteria such as Escherichia coli (E. coli), Staphylococcus aureus (S. aureus), or P. aeruginosa and inhibits 99% of bacterial growth.

Su et al. Polymers 2019, 11, 1050, discloses that eye contact lens preservatives are effective in cleaning, storing, and disinfection. But the contents of the preservatives may cause immune reactions, leading to eye conditions, including papillary conjunctivitis, peripheral ulcer, and superior limbic keratoconjunctivitis. The study revealed that γ-PGA could replace eye contact lens preservatives. γ-PGA is also significantly less harmful to the eye compared to typical contact lens solutions as it acts as a lubricative non-toxic antimicrobial agent that can reduce immune reactions from eye conditions and combat bacteria such as P. aeruginosa and C. albicans, common microorganisms that are present on contact lenses. The biopolymer has been used effectively as a solution in this in-vitro.

US 2010/0256050 to Sung et al., which is incorporated herein by reference, disclose pharmaceutical compositions containing poly-gamma-glutamic acid γ-PGA for inhibiting or preventing viral infection. US 2013/0267463 to Youn et al., which is incorporated herein by reference, disclose using γ-PGA for use in preventing or treating Th17 mediated diseases such as multiple sclerosis, rheumatoid arthritis, systemic lupus erythematosus, inflammatory bowel disease, Crohn's disease, and ankylosing spondylitis. US 2016/0158274 to Lee et al., which is incorporated herein by reference, disclose using γ-PGA in treating mucus hypersecretory disease. γ-PGA is commercially available such as from Vedan Enterprise Corporation, Taichung, Taiwan. U.S. Pat. No. 7,364,875 to Prescott, which is incorporated herein by reference, discloses methods of producing high molecular weight γ-PGA.

The oral and nasal wash of the present invention is particularly suited for patients who have wounds or lesions in the oral cavity and/or nasal passages. These especially include patients who have undergone cleft lip and palate surgery.

For example, cleft lip and palate (CLP) is a medical condition where babies are born with an unfused lip and/or palate due to a lack of tissues present to fuse the lip/palate together when the fetus grows in the womb. These surgeries include fusing the lip and palate together, rhinoplasty for deviated septa, etc. During recovery, patients are often unable to brush their teeth, floss, or use chemical oral and nasal washes to allow wounds to recover properly. The best alternative is to use saline nasal and oral solutions, which are not ideal for killing most bacteria left after surgical procedures. Most children with CLP are prone to infection in the ears, nose, and mouth, and are exposed to even more bacteria in post-surgical recovery. For example, children with a cleft palate are more prone to ear infections because the Eustachian tube in the ear cannot properly drain any fluid from the inside of the ear out into the back of the nose. This is caused by the unusual position of the muscles and tendons in the cleft palate. Additionally, microorganisms can harbor in the wounds after CLP surgeries have been performed. CLP patients often endure further bacterial infections and diseases when many bacteria often fail to be killed with saline oral and nasal washes, including periodontal diseases and bacterial infections (e.g. Staphylococcal and Streptococcal) and even Pseudomonas.

The oral and nasal wash of the present invention preferably comprises an aqueous composition typically containing 96,000 to 104,000 ppm and preferably 100,000 ppm or higher of γ-PGA. 100,000 ppm of γ-PGA in the nasal and oral washes can sufficiently kill almost all bacteria found in the mouth and sinuses unlike saline washes and is nontoxic to the body. The product can be obtained in a bottle (oral) or nasal spray bottle.

The molecular γ-PGA for the present invention typically ranges from 350 to 450 kDa and preferably 400 kDa.

The aqueous composition of the present invention can also include a solution of sodium chloride 0.65% in purified water (USP), made isotonic with sodium phosphate dibasic and sodium phosphate monobasic, with benzyl alcohol and benzalkonium chloride as preservatives; it will be used as the base solution for 100,000 ppm γ-PGA.

In using the aqueous composition as an oral or mouth wash, a person can empty a small quantity (such as 4 to 8 fluid ounces) of the aqueous composition into his or her mouth and swish the liquid around. While rinsing, the person can also gargle to wash the throat.

In using the aqueous composition as a nasal rinse, a person can use a squeeze bottle, bulb syringe, or neti pot, to pour or squeeze the liquid slowly into the upper nostril while tilting the head to one side. The liquid will pour out the other nostril. The person should breathe through his or her mouth and not the nose, at this time. Repeat on the opposite side. Besides using this nasal irrigation technique, the aqueous composition can also be applied directly into the sinus with a nasal swab.

The foregoing has described the principles, preferred embodiments and modes of operation of the present invention. However, the invention should not be construed as being limited to the particular embodiments discussed. Thus, the above-described embodiments should be regarded as illustrative rather than restrictive, and it should be appreciated that variations may be made in those embodiments by workers skilled in the art without departing from the scope of the present invention as defined by the following claims.

What is claimed is:

1. An oral or nasal wash composition comprising an aqueous solution containing γ-PGA.

2. The composition of claim 1 wherein the γ-PGA has a molecular weight range 350 to 450 kDa.

3. The composition of claim 1 wherein composition contains 96,000 to 104,000 ppm γ-PGA.

4. The composition of claim 1 wherein the γ-PGA inhibits or kills microorganisms.

5. A method of preventing infections in the nasopharyngeal cavity in a person that comprises administering a pharmaceutically effective amount of poly-gamma-glutamic acid (γ-PGA).

6. The method of claim 5 wherein the γ-PGA has a molecular weight range 350 to 450 kDa.

7. The method of claim 5 comprises administering an aqueous composition containing γ-PGA.

8. The method of claim 7 wherein the composition contains 96,000 to 104,000 ppm γ-PGA.

9. The method of claim 7 comprising rinsing the nasopharyngeal cavity by applying a fresh composition through one nostril and allowing a rinsed composition to exit the other nostril.

10. The method of claim 5 wherein the person suffers from a cleft lip and palate.

11. The method of claim 5 wherein the γ-PGA inhibits or kills microorganisms.

12. A method of preventing infections in the oral cavity of a person that comprises administering a pharmaceutically effective amount of poly-gamma-glutamic acid (γ-PGA).

13. The method of claim 12 wherein the γ-PGA has a molecular weight range 350 to 450 kDa.

14. The method of claim 12 comprises administering an aqueous composition containing γ-PGA.

15. The method of claim 14 comprising rinsing the person's month with the aqueous composition.

16. The method of claim 12 wherein the composition contains 96,000 to 104,000 ppm γ-PGA.

17. The method of claim 12 wherein the person suffers from a cleft lip and palate.

18. The method of claim 12 wherein the γ-PGA inhibits or kills microorganisms.
